(19) United States
(12) Patent Application Publication
(10) Pub. No.: US 2025/0160728 A1
(43) Pub. Date: May 22, 2025

(54) METHOD OF DIAGNOSING AND PREDICTING ALZHEIMER’S DISEASE PROGRESSION

(71) Applicants: Tapan Kumar Khan, Morgantown, WV (US); Subhrangshu Bit, Morgantown, WV (US)

(72) Inventors: Tapan Kumar Khan, Morgantown, WV (US); Subhrangshu Bit, Morgantown, WV (US)

(21) Appl. No.: 18/513,754

(22) Filed: Nov. 20, 2023

(51) Int. Cl. A61B 5/00; G16H 50/50

(52) U.S. Cl. CPC A61B 5/4088; A61B 5/7275; G16H 50/50

(57) ABSTRACT

The present invention relates to a method for diagnosing and predicting the course of Alzheimer's disease (AD)-related dementia after feature extraction of brain images by Variational Autoencoders (VAE) and using Artificial intelligence and Machine learning (AI/ML) algorithms, as compared with non-demented control cases (CN), mild cognitive impairment (MCI), and other non-Alzheimer's dementia (non-ADD) cases. Method 1 extracts the reduced dimensional latent feature vector from the sMRI scans/brain images using a VAE and does multiple sampling from the latent distribution to reduce the class imbalance followed by classification using different AI/ML models. Method 2, the extracted feature vectors from VAE are used as input to a mixture of class-Restricted Boltzmann Machines (cl-RBM) for AD, MCI, and CN classification. Method-1 diagnoses AD and distinguishes it from MCI, CN, and non-ADD using MRI scans with or without additional aids. Method-2 further extends to diagnosis and prediction of AD.

### FIELD OF INVENTION

The invention belongs to the field of neuroimaging diagnosis of Alzheimer's disease and distinguishing it from mild cognitive impairment, non-demented control cases, and other non-Alzheimer's dementia using MRI scans and machine learning-based algorithms with or without additional aids.

### BACKGROUND OF THE INVENTION

The present invention relates to Alzheimer's disease (AD) and, more particularly, to a method of diagnosing and predicting Alzheimer's disease progression from mild cognitive impairment and differentiating it from other non-Alzheimer's dementia patients.

Alzheimer's disease (AD) is an irreversible, slowly progressive neurodegenerative condition with no well-established disease-modifying therapy. Worldwide, approximately 50 million people are living with AD, and estimates show that this number will reach 152 million in 2050 [7]. Detection of the probable progression to AD from the preclinical AD or mild cognitive impairment (MCI) stage may provide a crucial window of opportunity to intervene with disease-modifying therapy. Alzheimer's disease (“AD”) has long been the subject of considerable efforts to develop accurate diagnostic methods and detect it at the earliest stage and progression. Despite these efforts, there is an unmet need for methods of accurately diagnosing AD, mild cognitive impairment (“MCI”) stage and differentiating it from non-Alzheimer's dementia (“non-ADD”). Prediction of Alzheimer's disease (AD) at the early stage, when the onset of symptoms begins without any clinical manifestation, is a significant clinical challenge and an excellent opportunity for early intervention to control further disease progression [1]. AD prediction has been attempted using different biomarker modalities. Still, research outcomes on AD prediction in the mild cognitive impairment (MCI) stage using neuroimages combined with cerebrospinal fluid (CSF) and genetic biomarkers are expensive, time-consuming, and unreliable. As a result, those are impractical predictors of AD progression [5].

Early detection of AD is ultimately required for therapeutic intervention and patient stratification for clinical trials. Almost all clinical trials for AD drug discovery failed recently. One of the critical causes is selecting patients at the advanced AD stages with widespread irreversible neuronal damage. The pathology of AD occurs as a sequence of events that start years or decades before clinical dementia appears. A prolonged phase of preclinical AD has been described in numerous studies, followed by mild cognitive impairment (MCI). Identification of individuals in the preclinical phase of AD would provide the best window of opportunity for therapeutic intervention to slow the progression of the disease. Preclinical AD is the stage to receive the best possible care, slowing the disease progression. Therapeutic interventions are currently focused on advanced AD dementia, and most of the clinical trials of these therapies have failed. The preclinical stage is challenging to detect-however, later stages of AD, like mild cognitive impairment (MCI), are detected by biomarkers. Detection of MCI by minimally invasive peripheral biomarkers holds an enormous promise for identifying individuals with MCI who can develop AD and find the risk of development of AD dementia in the future.

The current standard of care (SOC) tests to diagnose AD have many limitations, including lack of accuracy, subjectivity, limited coverage, high costs, and often a lack of a definitive diagnosis [5, 8, 9, 12]. Only 48% of physicians are satisfied with current SOC tests to diagnose AD. The current diagnostic pathway may lead to frustration and anxiety for patients and caregivers and negatively impact patient management. A test providing an earlier, more definitive, and accurate diagnosis would help physicians provide more supportive care, prescribe appropriate treatments, and enroll patients in clinical trials, where applicable. The indirect and direct cost burden of treating and managing AD and dementia is significant. More than 99% of AD drug trials failed in several decades. Reasons for failure are selecting wrong targets, biological heterogeneity of disease mechanisms, and poor patient selection. An ideal trial design should have a placebo-AD arm with a measurable cognitive decline due to AD-related brain pathology changes faster than the treatment arm.

Two central problems with detecting Alzheimer's dementia are diagnosis or classification of the stages (whether non-demented control (CN), mild cognitive impairment (MCI) or Alzheimer's disease (AD)) and forecasting of the disease progression (CN to MCI, MCI to AD, CN to AD), both of which are done using clinical and neuroimaging data (Khan 2016; Fisher et al., 2019). Machine Learning (ML) and Deep Learning (DL) techniques such as SVM, ANN, CNN, Autoencoder, Boltzmann Machines, etc., are found to be very useful for the diagnosis of Alzheimer's disease (AD) (Moradi et al., 2014, 2015; Pellegrini et al., 2018). The classification accuracy, however, depends on the type of problem (CN vs. AD/CN vs. MCI/MCI vs. AD). The best classifiers can discriminate between CN and AD subjects with accuracies in the mid-90% range. Still, they have considerably lower accuracies when discriminating between control and MCI subjects or between MCI non-converter (MCI-nc) to AD and MCI converter (MCI-c) to AD subjects. The best classifiers combine optimum features from different modalities, including CSF biomarkers, MRI, FDG-PET, cognitive measures, and factors such as age and APOE4 allele status (Weiner et al., 2015).

Machine learning (ML) and deep learning (DL) techniques, such as support vector machines, convolutional neural networks, autoencoders, etc., hold significant potential in the diagnosis or classification of the stage of AD dementia. However, forecasting models of AD progression tend to be more complicated because of the multi-chotomous progressive stages, and the time of such progression is also uncertain. The ambiguity in determining the clinical stages due to the heterogeneity of symptoms and patient conditions is an added complexity.

Combinations of sMRI and PET or other biomarkers have shown the potential to model such progression. Only very specialized centers that meet all infrastructure and regulatory compliance requirements and have highly technically trained expert teams of neuroscientists, radiologists, imaging equipment, and bioinformatics specialists can perform PET imaging. Maintenance for PET radiotracers with short half-lives (e.g., 11C-PIB with a half-life of ˜20 min) and ready access to a cyclotron are also required [5]. For these reasons, PET AD biomarkers are more costly and geographically limited than sMRI.

As can be seen, there is a need for methods of accurately diagnosing AD, mild cognitive impairment stage, and differentiating it from non-Alzheimer's dementia and a test providing an earlier, more definitive, and accurate diagnosis.

The present invention provides a platform that solves these needs using a comprehensive deep learning and probabilistic graphical model that leverages the neuroimaging information extracted from a subject's structural MRI (sMRI) scan. This can cluster the low dimensional representations of sMRI scans that allow investigation of the longitudinal effect and predict the possible temporal transition to advanced stages of AD.

The platform addresses early and accurate diagnosis of AD, a significant unmet need. The technology has a substantial commercialization potential for targeted market segments and can disrupt the brain imaging biomarkers technology for other diseases. AI/ML-based image analysis platform is a superior market fit and fits the current company setup. The present invention targets two major marketing segments with this software platform: the clinical market and the AD drug development industry.

The clinical markets comprise radiologists, neurologists, and geriatric psychologists' medical centers with MRI facilities, as well as clinical settings such as hospitals and neurology centers. The clinical market benefits from early detection of AD converted from MCI using only MRI scans. Confirmed results help the caregiver with the right plan. The clinical market also benefits from accurate diagnosis and prediction of AD progression using MRI scans and current analysis platforms.

The second stakeholder in this market segment is the AD drug development industry to stratify the suitable MCI cases to convert to AD. Proposed drugs have a better chance of producing positive outcomes if applied before the widespread neuronal loss. Therefore, the MCI stage is the right state for patient selection. AD drug trials focus on a newer strategy to conduct trials on Mild Cognitive Impairment (MCI) patients. The early-stage AD patient selection before widespread neuronal loss is critical for AD drug development using MRI data. The present invention helps patient stratification in AD clinical trials.

The current invention can disrupt the targeted market segments. NIA and AA incorporated s-MRI as one of the AD biomarkers to detect AD for research and clinical trial purposes in 2011. European Medicines Agency (EMA) has introduced MRI data as the primary biomarker data for patient stratification. Moreover, FDA clearance is optional for this marketing segment. Biolmaginix LLC's AI/ML-based image analysis platform has ˜90% accuracy, producing enormous value for payers, primary care physicians, patients, and the U.S. healthcare system in the battle to address AD efficiently and effectively.

### BRIEF DESCRIPTION OF THE DRAWINGS

FIG. 1 depicts a schematic flowchart illustrating a process for predicting and diagnosing Alzheimer's disease according to an embodiment of the present invention. sMRI 3-dimensional scans are registered as input, followed by skull stripped, convolutional neural network (CNN)-based segmentation, and normalized. Inside the dotted box, in the first step, processed MRI scans are used as input in the Variational Autoencoder (VAE) to produce the output of latent feature vectors. In the second step, the latent feature vectors are used in different machine learning (ML) models to diagnose Alzheimer's disease and distinguish it from mild cognitive impairment, non-demented control cases, and other non-Alzheimer's dementia using MRI scans with or without additional aids.

FIG. 2 depicts a schematic illustration of the flow diagram of the process for predicting the progression of Alzheimer's disease (AD) from an MRI scan using a mixture of class Restricted Boltzmann Machine (cIRBM). High-dimensional 3D sMRI scans were incorporated as input. The processing steps of MRI images consist of registration, skull stripping, and normalization. Processed images are used as the input in Variational Autoencoder (VAE) to extract reduced dimensional latent feature vectors from the sMRI scans. Inside the dotted box, in the first step, processed MRI scans are used as input in the Variational Autoencoder (VAE) to produce the output of latent feature vectors. In the second step, the latent feature vectors are used in using a mixture of class Restricted Boltzmann Machine (cIRBM) to diagnose Alzheimer's disease and distinguish it from mild cognitive impairment, non-demented control cases, and other non-Alzheimer's dementia using MRI scans with or without additional aids. Schematic diagram of the VAE-mcIRBM model for predicting Alzheimer's progression: A. Variational Autoencoder: Generates a latent representation (z) of a MRI scan (X). fϕ, gψ are the encoder and decoders respectively with ϕ being the variational parameters and w being the generative parameters. (μ, σ) are the parameters of the generated latent distribution. B. Mixture of Class-RBM: Generates conditional transition probability matrix which gives the probabilistic progression from current stage to advanced stages of AD. qk is the activation variable determining the mixture component and the current state of Alzheimer's dementia. Gk, Wk. are the parameters of the class RBM. h is the hidden vector and y represents the future state of Alzheimer's.

FIG. 3 depicts Variational Autoencoder (VAE) image processing steps consisting of an encoder and a decoder in a latent Gaussian model where all are parametrized by a generative Convolutional Neural Network (CNN) with the computer architecture of a variational autoencoder and decoder utilized. ReLU=Rectified Linear Unit.

FIG. 4 depicts an example of a preprocessing pipeline on typical sMRI scans: (A) Space normalization ensures that the spatial structure of all the images of the dataset is as similar as possible with each scan resampled to a (1×1×1) mm3 of voxel space. (B) Registration adapts the sMRI scan to another reference image, which is called an atlas (we use T1 weighted MNI152), seeking that the same regions of both represent the same anatomical structure. (C) Skull stripping removes the information from the skull that appears on structural MRI images. (D) Min-max normalization reduces the pixel values varying between 0-1 to maintain similar pixel variation among the sMRI scans.

FIG. 5 depicts a schematic view of cluster identification by t-distributed Stochastic Neighborhood Embedding (t-SNE) of the latent vectors. It shows the projection of Variational Autoencoder (VAE) latent dimensions. The reduced dimensional latent representation of MRI scans generated using VAE is further reduced to two dimensions to visualize the class-wise embeddings and clusters. A: CN-AD; B: CN-LMCI; C: AD-LMCI (Feature [1]: t-SNE embedded dimension 1 and Feature [2]: t-SNE embedded dimension 2; AD, Alzheimer's disease; CN, Control; LMCI, Late Mild Cognitive Impairment).

FIG. 6 depicts distributed Stochastic Neighborhood Embeddings (t-SNE) of the latent vectors. It shows the projection of Variational Autoencoder (VAE) latent dimensions. The reduced dimensional latent representation of MRI scans generated using VAE is further reduced to two dimensions to visualize the class-wise embeddings and clusters. The spherical nature of the clusters indicates that the samples are generated from a normal distribution. Significant upsampling from a small number of data points in class Advs. PD results in densely populated green patches. (Feature [1]: t-SNE embedded dimension 1 and Feature [2]: t-SNE embedded dimension 2; AD, Alzheimer's disease; PD, Parkinson's disease).

FIG. 7 depicts the performance of classification of Alzheimer's disease (AD) versus non-demented Control (CN). A. Performance of different machine learning models (Ridge Classifier, Light Gradient Boosting Machine, Extra Trees Classifier). B. The Receiver Operating Characteristic (ROC) is traced from the true positive rate and false positive rate of prediction.

FIG. 8 depicts the performance of classification of Autopsy-Confirmed Alzheimer's disease (Autopsy-AD) versus non-demented Control (CN) A. Performance of different machine learning models (Ridge Classifier, Light Gradient Boosting Machine, Extra Trees Classifier). B. The Receiver Operating Characteristic (ROC) is traced from the true positive rate and false positive rate of prediction.

FIG. 9 depicts the performance of classification of non-demented Control (CN) versus Late Mild Cognitive Impairment (LMCI). A. Performance of different machine learning models (Ridge Classifier, Light Gradient Boosting Machine, Extra Trees Classifier). B. The Receiver Operating Characteristic (ROC) is traced from the true positive rate and false positive rate of prediction.

FIG. 10 depicts the performance of classification of Alzheimer's disease (AD) versus Late Mild Cognitive Impairment (LMCI). A. Performance of different machine learning models (Ridge Classifier, Light Gradient Boosting Machine, Extra Trees Classifier). B. The Receiver Operating Characteristic (ROC) is traced from the true positive rate and false positive rate of prediction.

FIG. 11 depicts the performance of classification of Alzheimer's disease (AD) versus Parkinson's disease (PD). A. Description of the setup. B. Performance of different machine learning models (Extra Trees Classifier, Light Gradient Boosting Machine, Random Forest Classifier, and Extreme Gradient Boosting). C. The Receiver Operating Characteristic (ROC) is traced from the true positive rate and false positive rate of prediction.

FIG. 12 depicts the probability of a specific diagnosis, Alzheimer's disease (AD), Late Mild Cognitive Impairment (LMCI), Early Cognitive Impairment (EMCI), and non-Demented Control (CN) in terms of Alzheimer Score. The machine learning model predicts a specific Alzheimer Score from an MRI scan.

FIG. 13 depicts two variants of Boltzmann Machines. (A) Classification Restricted Boltzmann Machine. The condition z contains the vector representation of the image of interest. The hidden and output variable set forms a bipartite graph representing Boltzmann structure with conditional independence. (B) Mixture of Conditional class-Restricted Boltzmann Machine is a combination of such class restricted Boltzmann machines with an activating variable deciding the set of parameters.

### DETAILED DESCRIPTION OF THE INVENTION

The encoder in VAE, consisting of 3D CNN blocks, plays a pivotal role in compressing the input sMRI data into a lower-dimensional representation, commonly known as a latent space. Its primary objective is to discern and encapsulate the critical features and intricate patterns inherent in MRI brain images. This encoding process serves as the foundation for various downstream tasks such as data compression, feature extraction, and denoising. To ensure that the model effectively captures the underlying structures in sMRI brain images, various data augmentation techniques, including translation, scaling, rotation, flipping the 3D image around an axis, and the addition of Gaussian noise, were applied with specific probabilities. The decoder in the current MRI autoencoder, mirroring the encoder, contains 3-D CNN blocks and aims to reconstruct the compressed latent space back into the original MRI data format. It employs a symmetrical architecture to the encoder, reversing the encoding process to generate output images that closely resemble the non-augmented original MRI data. The loss function incorporates both the mean squared error (MSE) term and the Kullback-Leibler (KL) divergence term. The MSE term is calculated within the masked region (the region where the brain tissue is present in the original image) of both the model's output and the original image, measuring pixel-wise reconstruction fidelity. Simultaneously, the KL divergence term is instrumental in regularizing the latent space representations, encouraging them to follow a specific distribution, typically a Gaussian distribution with a mean of zero and a standard deviation of one. This dual-loss approach ensures both accurate reconstruction and effective latent space regularization, contributing to the overall model's robustness and performance in handling MRI brain data. Our novel approach using a VAE excels at extracting crucial features from MRI brain images in an unsupervised fashion. VAE has been used in brain tumor 3D MRI image segmentation [11]. By incorporating the VAE framework here, the model learns to capture the inherent structure and salient features of the data without the need for explicit feature labeling or supervision. This unsupervised learning process is especially advantageous in MRI data, where the identification of crucial features can be complex and multifaceted. The VAE's ability to implicitly discover and represent these features within the latent space not only streamlines the modeling process but also opens the door to finding previously unknown or subtle patterns and information within the MRI data, ultimately enhancing our understanding of the underlying neurobiology.

The following detailed description is of the best currently contemplated modes of carrying out exemplary embodiments of the invention. The description is not to be taken in a limiting sense but is made merely to illustrate the general principles of the invention since the appended claims best define the scope of the invention.

Although the present specification exemplifies the results of the model on a few cases of disease progression, the model is generalized and may be utilized in various other modalities and tasks. While the current examples use only sMRI scans as input, the model allows the usage of any other specific modality, such as fMRI, PET, etc., or the combination of different modalities, such as clinical and genetic data. Moreover, this method is not restricted only to the progression of Alzheimer's but can be extended to use on other kinds of disease progression as well. For example, the invention may also be used to find brain tumors and brain lesions in Multiple sclerosis (MS).

Extracting relevant features from the vast amount of complicated information in sMRI scans is an important step. Rather than producing a single point prediction under the unimodal assumption, the inventors attempted to approximate the overall distribution of future disease progressions, i.e., CN to MCI, MCI to AD, CN to AD. To tackle the complicated data distributions of 3D MRI scans, a two-part generative hybrid neural network model, a self-supervised (VAE) and supervised learning (cl-RBM), addresses AD progression uncertainty. The generative nature of the model allows sampling from the predicted probability distribution to generate synthetic patient information that can ameliorate the unavailability of medical records of a larger cohort attributed to similar characteristics.

The inventive AI/ML platform, unlike most of the existing methodologies, is operative to accurately determine the current stage of dementia (CN (non-demented control)/MCI/AD) and to predict the probabilistic progression to an advanced stage based only on 3D structural brain imaging (sMRI) scans. The image analysis platform of the present invention generally uses 3D structural MRI (sMRI) scans from two internationally recognized datasets (ADNI and OASIS-3e) as input.

With the present invention, 3-D MRI scans may successfully diagnose and predict the course of AD-related dementia. Predictions of the current and future state of a patient's AD may be computed from the model by generating latent representations of the patient's sMRI scan (current). The two significant challenges of using 3D MRI scans are high-dimensional images and a low number of scans [high dimensional low sample size (HDLSS)], a common problem in all medical data. These challenges have been overcome using a modified version of a Variational Autoencoder (VAE) [3] to extract the reduced dimensional latent feature vector from sMRI scans after performing spatial normalization, skull extraction, and Minmax normalization. A low-dimensional latent space conditioned on the current MRI scan is modeled through the VAE. The data is high dimensional (˜9 million pixels) and involves complicated dependencies, while the latent representation lies in a much lower dimensionality than that of the data space. This part of the model captures the internal stochasticity from the data and generates latent representations of a subject's MRI. Since the modeled low dimensional space is generated in a probabilistic setting, it allows sampling from the data set to generate new synthetic patients' information. Multiple sampling is then performed from the latent distribution to reduce the class imbalance among training samples. The tSNE (t-Stochastic Neighborhood Embedding [10]) embeddings of different classes form three distinct clusters. Sampling from the generated conditional distribution helps augment the dataset. The ability to sample from any conditional distribution is one advantage a modeling framework based on Variational Autoencoders and cl-RBMs has over alternative discriminative models.

The Variational Autoencoder (VAE) extracts relevant features from sMRI scans of subjects using a self-supervised learning method. The features extracted from the VAE are fed as inputs into a generative deep neural network that is a novel mixture of class-restricted Boltzmann Machines (cl-RBMs) and other classifiers (e.g., Ridge Classifier, Light Gradient Boosting Machine, Extra Trees Classifier). An RBM is a special kind of Markov random field that is represented as an undirected bipartite graph. RBMs can be made to fit various datasets by varying the number of nodes in the graph, but for those with several distinctly different types of data, such as images of other object classes, mixture models are more accurate. The output is a conditional transition probability matrix that provides the complete picture of future progression probabilities.

The inventors discovered a methodology that leverages the power of Variational Autoencoders (VAEs) to extract latent distributions from sMRI and the generative power of RBMs to predict the distribution of disease progression. The mixture of class Restricted Boltzmann Machines (cl-RBM) may determine the optimal model parameters for three separate classes of dementia (CN, MCI, and AD) by maximizing a regularized conditional log-likelihood with added constraints that ensure that the input condition latent representation belongs to only one of the three stages of dementia and progression happens to only advanced stages.

The invention introduces a series of steps in an AI/ML algorithm, incorporating two logics (e.g., autoencoder and one of Ridge Classifier, Light Gradient Boosting Machine, Extra Trees Classifier, Boltzmann machine) together to achieve better performance. The inventive algorithm may be used to distinguish AD from other non-AD dementias (e.g., multi-infarct dementia, dementia due to Parkinson's disease, Lewy Body dementia, frontotemporal dementia, dementia due to Vitamin B12 deficiency, supra nuclear palsy, tauopathy, Frontotemporal Lobar Degeneration with TDP-43-Positive Inclusions, hippocampus sclerosis, etc.) using region-specific MRI scans. Moreover, brain autopsy studies found more than 50% of cases of AD pathologies remain comorbid with other forms of neurodegeneration. The present disclosure contemplates the introduction of a more specific argument base and brain-region-specific differentiating parameters for how AD pathology can be distinguished in the presence of another dementia.

The platform identifies and leverages a unique distinguishing factor in AD progression, i.e., the three separate classes of dementia (CN, MCI, and AD) have intrinsically different brain structures. The mixture of class RBMs captures this distinctiveness through a particular set of model parameters for each class. Assuming a subject cannot regress to a previous stage in dementia, the model ensures that a subject predicted with AD in the current state can only be in the same stage at a future time point. Similarly, for MCI, the only possible future states are MCI and AD. This allows the model to reduce the complexity of modeling the progression with fewer possibilities, thereby increasing the accuracy of diagnosis. However, while dealing with only three stages of AD, the number of parameters is significantly large. It could be a bottleneck due to the small amount of medical data with a larger number of stages. Finer stages with not-so-distinct characteristics, if modeled separately as a mixture component for each, may tend to overfit and are likely to be non-robust.

### Definition

The term “Alzheimer's disease” or AD refers to a condition where amyloid beta and phosphorylated tau deposit into some regions of the brain, and the patients have dementia due to brain atrophy. Besides atrophy, physical and functional changes occur in the brain due to the onset of the disease. There are two different kinds of AD, e.g., sporadic or late-on-set AD and familial or early-onset AD. AD is an irreversible disease condition, a slowly progressive neurodegenerative condition with no well-established disease-modifying therapy.

The term “Mild Cognitive Impairment” “MCI” refers to a condition intermediate between preclinical dementia and full-grown dementia. It can be divided into two categories: amnestic MCI (aMCI) and multimodal MCI (mMCI) or non-amnestic MCI. MCI with primarily memory deficits is called amnestic MCI. Multimodal MCI includes MCI with problems in thinking skills, inability to make sound decisions and judgments, and failure to take the sequential steps needed to perform relatively complex tasks. In general, individuals with aMCI eventually develop AD, and those with mMCI develop non-AD dementias. MCI has been divided into two categories based on clinical criteria, such as Mini-mental score examination (MMSE) and Clinical Dementia Rating (CDR), Late MCI (LMCI), and Early MCI (EMCI) (Table 1). MCI proceeds to AD with an annual rate of 10-12% (Petersen et al., 1999), and an individual with MCI is expected to convert to AD within five years (about 5-25% per year) (Petersen et al., 2001). Another study found a slightly higher rate (10-25%) of conversion from MCI to AD (Grand et al., 2011). Patients with MCI who are progressing to AD have c-MCI, and those who continue to stay at the MCI stage have stable MCI (s-MCI). Individuals with c-MCI might be better candidates for inclusion in clinical studies of AD drugs to test their efficacy in slowing progression to moderate and severe AD.

Non-Alzheimer's dementia or “non-AD dementia” refers to dementia due to Lewy body dementia (LBD), vascular dementia (VaD), dementia due to frontotemporal dementia (FTD), dementia due to Parkinsonism, corticobasal degeneration (CBD), progressive supranuclear palsy (PSP), tauopathy, dementia due to vitamin B12 deficiency, etc. Clinical criteria for non-AD dementia include the loss of cognitive functions such as the ability to think, remember, or reason to the point that it is.

“Artificial Intelligence” or “AI” refers to a branch of science where a computer system performs intelligently by using data fed by humans in areas such as decision-making, image recognition, intuitive design, language translation, and speech recognition. Here, the computer-driven machine acts intelligently like human intelligence and performs exceptionally better. There are four kinds of AI: theory of mind, limited memory, reactive limited memory, and self-awareness.

An “autoencoder” refers to a neural network architecture that can learn compact representations of data, making them a valuable tool in a wide range of machine learning and deep learning applications. It has two parts: Encoder and Decoder. The Encoder part of the network takes an input, which could be an image, text, or any other data, and maps it to a lower-dimensional representation. This lower-dimensional representation is often referred to as the “latent space” or “encoding.” The encoder network consists of one or more layers, typically reducing the dimensionality of the input data gradually. The Decoder part takes the lower-dimensional encoding and tries to produce the original input data. The decoder's architecture is essentially a mirror image of the encoder, and it increases the dimensionality of the encoding to match the original data's dimensionality.

“Variational Autoencoder” or “(VAE) is a type of generative model and an extension of the traditional autoencoder. VAEs are used for unsupervised learning and generative tasks, particularly in deep learning. What sets VAEs apart from standard autoencoders is their ability to generate new data points that are like the training data and to produce a continuous and structured latent space. A VAE works in the following way: Encoder: Like a traditional autoencoder, a VAE consists of an encoder network that maps input data to a lower-dimensional latent space. However, in a VAE, the encoder doesn't produce a single fixed encoding. Instead, it produces two vectors: a mean (μ) and a standard deviation (σ) that parameterize a probability distribution (typically Gaussian) in the latent space. Sampling: The key innovation in VAEs is the introduction of a stochastic element. Instead of a fixed encoding, VAEs sample a point in the latent space from the learned probability distribution. This sampling introduces a source of randomness, allowing VAEs to generate new data points. The current encoder in VAE, consisting of 3D CNN blocks, plays a pivotal role in compressing the input MRI data into a lower-dimensional representation commonly known as a latent space. Decoder: The decoder takes the sampled point from the latent space and reconstructs the original data. Objective Function: VAEs use a loss function that consists of two parts: a reconstruction loss, which measures how well the generated data matches the input data, and a regularization term based on the Kullback-Leibler (KL) divergence between the learned distribution in the latent space and a chosen prior distribution (usually a standard Gaussian). The regularization term encourages the latent space to be continuous and structured. During training, VAEs aim to minimize this combined loss. This process enables the encoder to learn meaningful representations in the latent space and ensures that the generated data points follow a distribution that allows for smooth interpolation and sampling. The VAE's ability to generate new data points from the learned latent space makes it useful for tasks like image generation, denoising, and interpolation. VAEs are also known for their generative capabilities and potential to produce novel data samples that resemble the training data distribution. VAEs are widely used in deep learning and have significantly contributed to fields like image generation, natural language processing, and generative modeling. They provide a way to learn continuous and structured representations of data that can be sampled to generate new, similar data points.

Conditional Restricted Boltzmann Machines (cRBM) refers to a multidimensional system network modeling that can learn a probability distribution over a set of closely associated data. It has been used in dimensionality reduction, classification, collaborative filtering, feature learning, and machine learning modeling. cRBM is a generative neural network model which has been used for forecasting multiple indicators of disease. In this invention, the mixture of class Restricted Boltzmann Machines (cl-RBM) can determine the optimal model parameters for three separate classes of dementia (CN, MCI, and AD).

### Description of Embodiments

In an embodiment, broadly, the present invention provides an image analysis platform based on an AI/DL model that can predict the conversion chance of non-demented cases to mild cognitive impairment (MCI) and MCI to AD. Another embodiment of the present invention provides a method for determining whether a human subject is afflicted with Alzheimer's disease (“AD”) or non-Alzheimer's dementia (“non-ADD”) when the subject is suspected of being afflicted with either AD or non-ADD.

In another embodiment, the probability of a specific diagnosis, e.g., Alzheimer's disease (AD), Late Mild Cognitive Impairment (LMCI), Early Cognitive Impairment (EMCI), and non-demented Control (CN) are predicted in terms of Alzheimer's Score. The machine learning model predicts a specific Alzheimer's Score from an MRI scan.

In a specific embodiment, VAE extracts latent distributions and inputs to specific machine learning classification, enabling us to determine the optimal model parameters for three separate classes of dementia (CN, MCI, and AD) that model outputs a conditional transition probability matrix (cTPM) that gives both the current stage and fed into ML classifiers.

In another embodiment, the extracted feature vectors from MRI images are used as input conditions to a mixture class Restricted Boltzmann Machines (cl-RBM) mixture model captures the distinctiveness of parameters of the three separate classes of dementia (CN, MCI, and AD).

In another specific embodiment, a methodology that leverages the power of VAEs to extract latent distributions and the generative power of RBMs. Our mixture of class Restricted Boltzmann Machines (cl-RBM) can determine the optimal model parameters for three separate classes of dementia (CN, MCI, and AD).

In a further embodiment, the cl-RBM model outputs a conditional transition probability matrix (cTPM) that gives both the current stage and probabilistic progression from the current stage to future stages of dementia, with a 70-30 (train and test) split of the total cases, we achieve an overall prediction accuracy of higher than 80%, with class-specific accuracies of 80% (CN), 70% (MCI), and 70% (AD) for current predictions and 70% (CN), 65% (MCI), 69% (AD) for future stage predictions.

### Methods

[ADNI Data: The ADNI dataset contains several collections of MRI images. In order to balance the number of examples in each of the classes, we made use of a collection of 204 MP-RAGE or MP-RAGE REPEAT scans. The images were divided into non-demented control (CN), Mild Cognitive Impairment (MCI), Alzheimer's disease (AD), Autopsy-Confirmed AD, and Parkinson's disease (PD).

Feature Extraction from MRI Scans Using Variational Autoencoder:

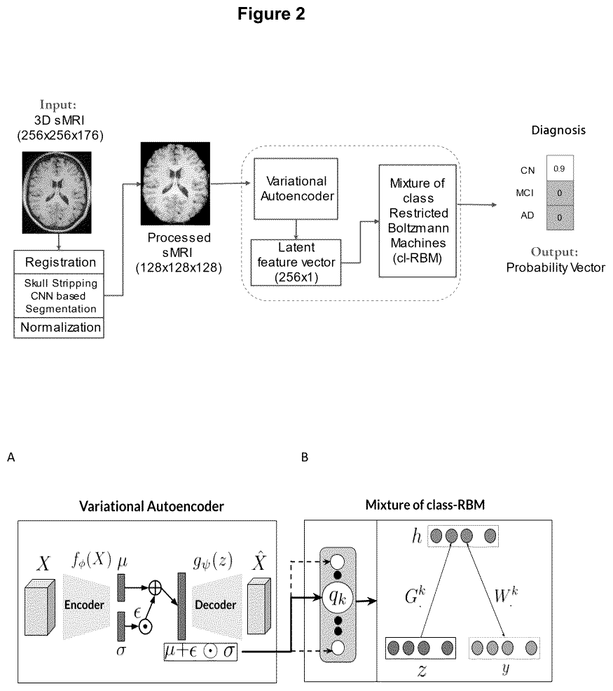

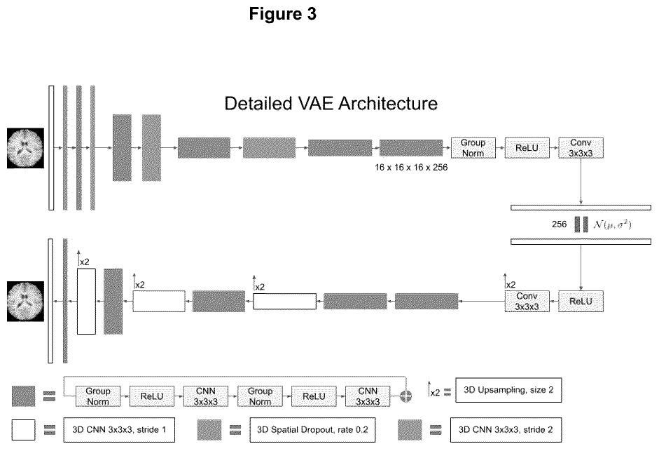

Extracting relevant features from the huge amount of complicated information present in MRI scans is an important step. Generative modeling is one type of unsupervised learning that deals with complicated data distributions. It could be interpreted as learning a generative process by which the observation data arose (Bishop, 2006). Feature extraction from MRI scans using Variational Autoencoder for this study has been presented in FIG. 2 and FIG. 3.

Encoder Part: The encoder part uses ResNet (He et al., 2016) blocks, where each block consists of two convolutions with normalization and ReLU, followed by additive identity skip connection. For normalization, we use Group Normalization (GN) (Wu and He, 2018), which shows better than BatchNorm performance when batch size is small (batch size is 1 in our case). We follow a common CNN approach to progressively downsize image dimensions by 2 and simultaneously increase feature size by 2. For downsizing we use stride convolutions. All convolutions are 3×3×3 with initial number of filters equal 32. The encoder endpoint has size 256 (128 for mean and 128 for variance).

Decoder Part. Starting from the encoder end point output, we first reduce the input to a low dimensional space of 256 (128 to represent mean, and 128 to represent standard deviation). Then, a sample is drawn from the Gaussian distribution N (z|μ_{ϕ}, Σ_{ϕ}). The sample is fed as input to the decoder structure which is like the encoder. Each decoder level begins with upsizing: reducing the number of features by a factor of 2 (using 1×1×1 convolutions) and doubling the spatial dimension (using 3D bilinear sampling). The end of the decoder has the same spatial size as the original image.

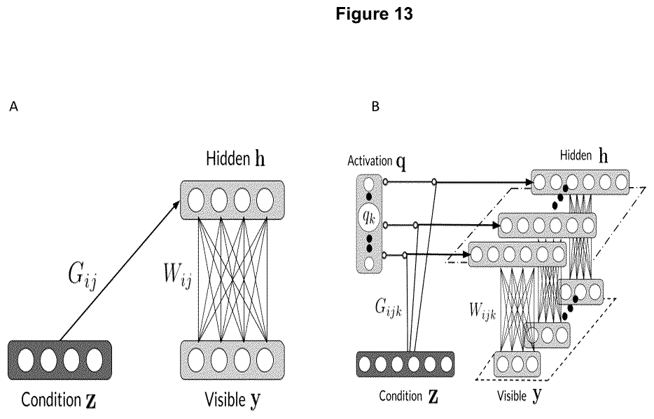

Variational Autoencoder and do multiple sampling from the latent distribution to reduce the class imbalance. The extracted feature vectors are then used as input conditions to a mixture class Restricted Boltzmann Machines (cl-RBM) mixture (FIG. 13). The mixture model captures the distinctiveness of parameters of the three separate classes of dementia (CN, MCI, and AD). Optimal model parameters are found by maximizing a regularized conditional log-likelihood with added constraints that ensure that the input condition latent representation belongs to only one of the three stages of dementia and progression happens to only advanced stages. The model outputs a conditional transition probability matrix (cTPM) that gives both the current stage and probabilistic progression from present to future stages of dementia.

### Example

AD vs. non-AD dementia was analyzed by collecting data from PPMI (16 PD) and ADNI (14 AD); registering all the images into one standard template (MNI152); stripping skull data using the FSL Brain Extraction Tool; and reducing the image to a latent representation of size 256 using the existing trained VAE to achieve an accuracy of 83%. The demographics of the subjects (206 total) used in the study were (a) Not Hispanic/Latino: 203; (b) Hispanic/Latino: 2; and (c) Unknown: 1.

For comparison, three baseline discriminative models were trained-Logistic Regression, Random Forest, and Support Vector Machine (SVM) that uses the latent representations of the sMRI scans generated from VAE to predict only the current state of Alzheimer's dementia. Note that a separate model is trained for each class (current state)—a total of 2 models for each of the discriminative classes. The hyperparameters are optimized using Bayesian optimization for each logistic regression and random forest model. In contrast, SVM was optimized using a grid search varying over different values of C, gamma, and Radial basis function (RBF)/polynomial kernel. The prediction of future states from AD was not considered based on an assumption that a subject cannot regress to a previous stage in dementia, i.e., that the only possible future state is AD. By contrast, a single implicit mixture of cl-RBM is used to predict the current and future states of Alzheimer's dementia.

The results demonstrate that the inventive algorithm utilizing the mixture of cl-RBM models achieves robust performance in predicting and diagnosing AD compared to the three machine learning models (Logistic Regression, Support Vector Machine and Random Forest) in terms of predicting the progression of AD. Future state predictions of AD using a mixture of cl-RBM were higher than logistic regression (LR), random forest (RF), and support vector machine (SVM) algorithms . . .

We have invented a new series of mathematical steps and procedures under U.S. patent law, which can be patentable. The reason this software/platform can be patented is that it's considered a finished product, whereas machine learning algorithms are considered abstract. It's now a finished product. The current invention serves a purpose—to be valid and not fall into the following categories: Laws of nature, Physical phenomena, or Abstract ideas.

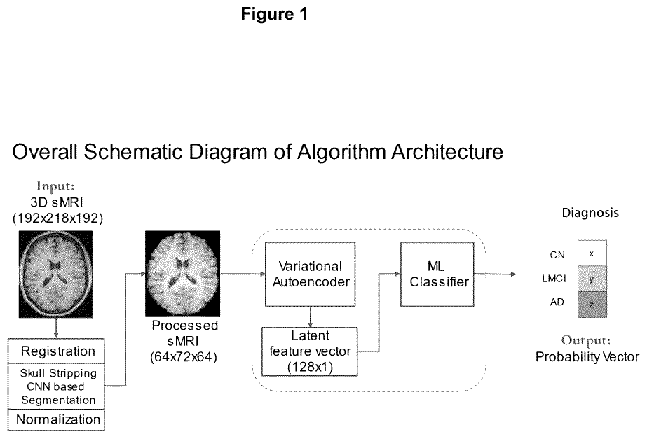

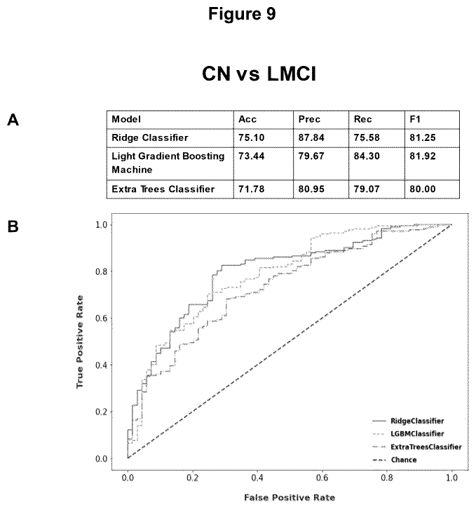

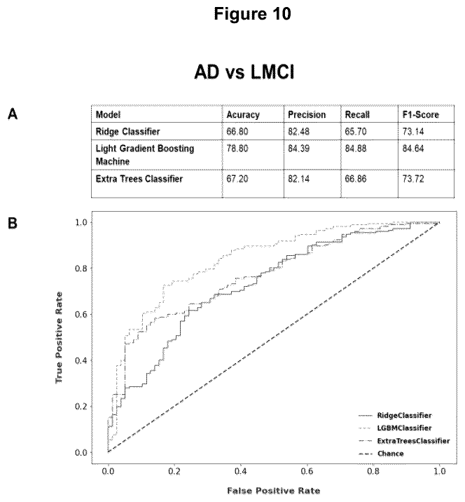

Referring now to FIG. 1 through FIG. 11:

FIG. 1 is a schematic flowchart illustrating a process for predicting the progression of Alzheimer's disease (AD) from an sMRI scan according to an embodiment of the present invention. The first step incorporates high dimensional 3D sMRI scans as input. The second step processes the MRI images by registration, skull stripping, and normalization to produce the processed images. As shown in FIG. 1, step generates reduced dimensional representations of the sMRI scans using a Variational Autoencoder and generates model parameters. In FIG. 2, A Mixture of Class RBMs is used in step to predict temporal progression in the form of a matrix shown in step, illustrating the prediction and progression from Control (CN) to Mild Cognitive Impairment (MCI) to Alzheimer's disease (AD).

FIG. 3 illustrates the function and description of Variational Autoencoder (VAE) image processing steps consisting of an encoder and a decoder in a latent Gaussian model where all are parametrized by a generative Convolutional Neural Network (CNN) with the computer architecture of a variational autoencoder and decoder utilized. ReLU=Rectified Linear Unit. In combination of VAE-m-cIRBM model utilized in the inventive method for predicting Alzheimer's progression. Section A represents a Variational Autoencoder, which generates a latent representation z of an MRI scan (X). Functions ƒ_{ϕ}, g_{ψ} are the encoder and decoders respectively; ϕ is the variational parameter; ψ is the generative parameter; and (μ, σ) are the parameters of the generated conditional latent distribution. These parameters are learnt by reducing the following loss function:

$$min - log {p}_{ψ} ( x ) + {L}_{{l}_{2}} ( x ,$$

where:
x: A patient's sMRI scan
p_{ψ}: Joint probability of all the patient's sMRI scan data
Lₗ_{₂}(x, \hat{x}): Euclidean distance between the original and the regenerated sMRI scan. Section B illustrates a mixture model of class-RBM. The model generates a conditional transition probability matrix which gives the probabilistic progression from current stage to advanced stages of Alzheimer's disease. The parameters include qk, the activation variable determining the mixture component and the current state of Alzheimer's dementia; h, which denotes the hidden vector; and y, which represents the future state of Alzheimer's. Gₖ and Wₖ are the parameters of the k^{th} class RBM. The joint probability distribution is modeled in the form:

$$p ( y , h , q \vert z ) = \frac{exp ( - E ( y , h , q \vert z ) )}{Z}$$

where, Z=Σ_{q} Σₕ Σ_{y} exp(−E(y, h, q|z)) is the normalizing constant, found by marginalizing over all possible hidden, observed, and initial activation labels. In case of a mixture model, the interdependency of hidden units h and visible units y is for an initial label q, given the latent or condition z. The energy term E defined as—

$$E ( y , h , q \vert z ) = - ( {\sum }_{i k} {b}_{i}^{k} {y}_{i} {q}_{k} - {\sum }_{j k} {c}_{j}^{k} {h}_{j} {q}_{k} - {\sum }_{k} {q}_{k} {\sum }_{i j} {W}_{i j}^{k} {y}_{i} {h}_{j} - {\sum }_{k} {q}_{k} {\sum }_{j l} {G}_{jl}^{k} {h}_{j} {z}_{l} )$$

The encoder uses convolutional operations along with normalization and rectified linear unit as activation function in the network. It follows a common approach of progressively downsizing the image by a factor of two at each layer to an end size of 256. The encoder end point is utilized as parameters to a latent distribution (assumed to be Normal). A sample drawn from this distribution is fed as input to the decoder. Each decoder level upsizes by reducing the number of features by a factor of two and doubling the spatial dimensions.

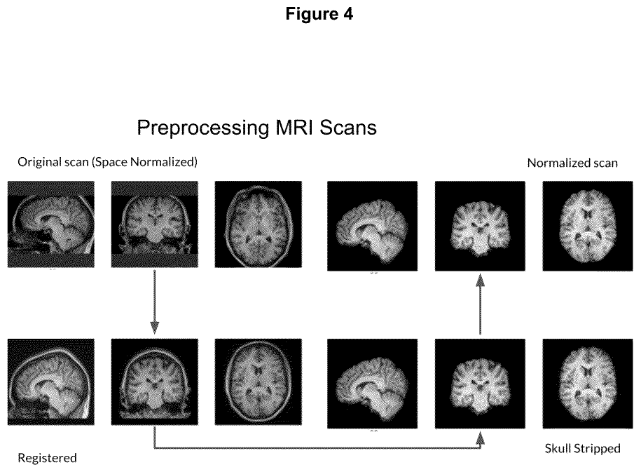

The steps of registration, skull stripping, and normalization are illustrated in FIG. 4 on typical sMRI scans. Column A illustrates the original scans, space normalized to ensure that the spatial structure of all the images of the dataset is as similar as possible, with each scan resampled to a (1×1×1)mm₃ of voxel space. As shown in column B, registration adapts the normalized sMRI scan to another reference image, called atlas (e.g., T1 weighted MNI152 template), so that the same regions of both represent the same anatomical structure. Skull stripping removes the information related to the skull that appears on structural MRI images, as shown in column C. The Min-max normalization of column D reduces the pixel values varying between 0-1 to maintain standard pixel variation among the sMRI scans.

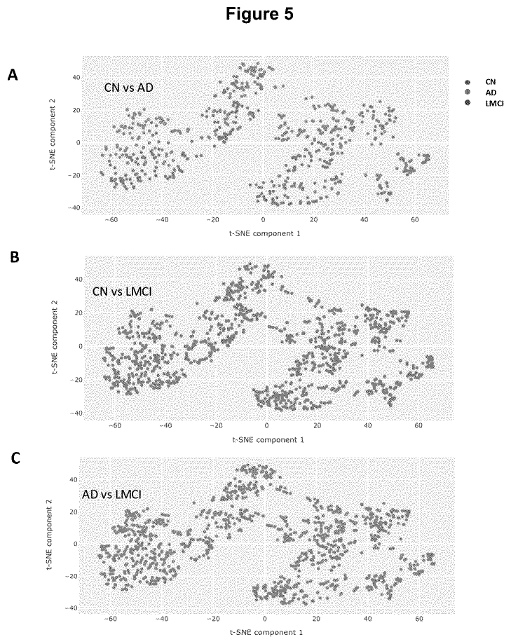

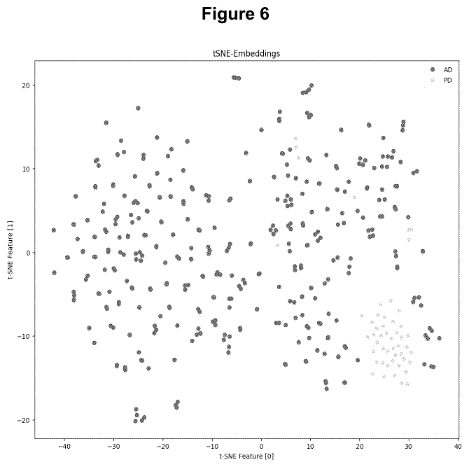

After multiple sampling, the t-distributed Stochastic Neighborhood Embedding (t-SNE) embeddings of different classes were found to form distinct clusters; see FIG. 5-6. Clustering results on the t-SNE plot show spherical patches of clusters indicating the samples are generated from a normal distribution. (Feature [1]: t-SNE embedded dimension 1 and Feature [2]: t-SNE embedded dimension 2.) Different clusters were identified from the latent features encoded by VAE with a hierarchical presentation.

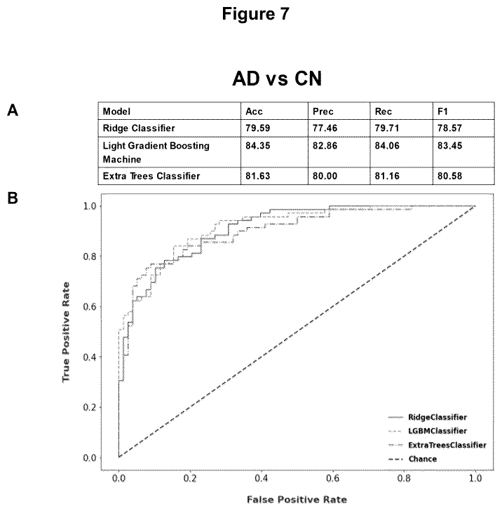

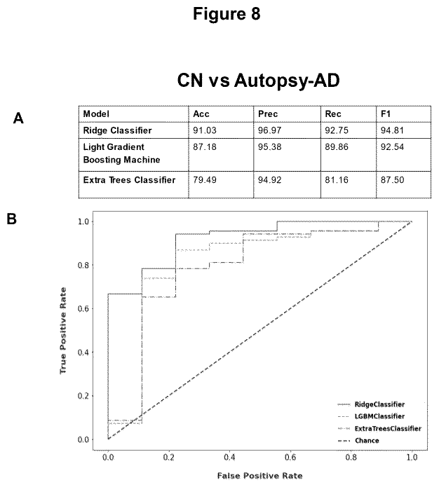

FIG. 7-10 and Table 3 and Table 4 illustrate the accuracy of classification by different machine leaning model when added with the VAE feature extraction and using most advanced machine learning models (Ridge Classifier, Light Boosting Machine, Extra Tree Classifier). Diagnostic characteristics are presented in terms of Accuracy, Precision, Recall, and F1-Score. Accuracy represents how accurate the ML algorithm classifies; Precision represents the correctness of the prediction; Recall represents the true positive percentage rate of prediction and F1 Score is the performance measure of the machine learning algorithm. It's a combination of Precision and Recall. F1-Score 1 indicates excellent precision and recall, while a low score indicates poor model performance. F-1 Score of 0.7 or higher acceptable performance. ROC curves indicate the magnitude of true positive rates. The best way to test an Alzheimer's disease diagnostic algorithm is to test it in autopsy-confirmed AD cases. Autopsy-confirmed AD is the National Institute of Health (NIH) “GOLD” standard confirmation of AD. The algorithm classified AD versus CN diagnosis with 91% accuracy, 97% precision, 93% Recall value and 0.95 F1 Score (FIG. 8).

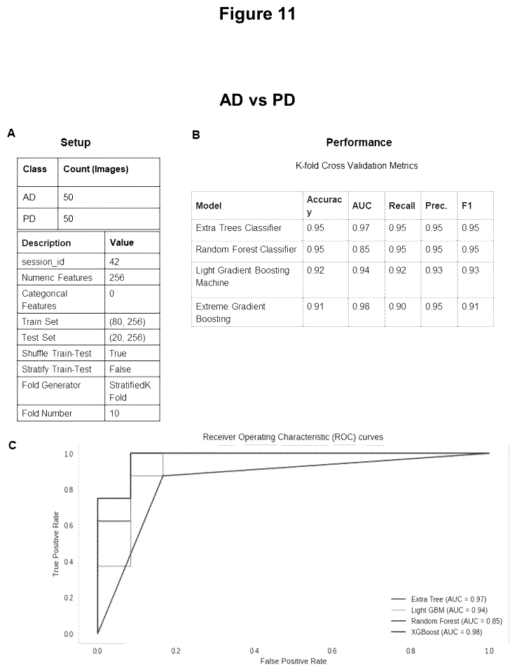

FIG. 11 illustrates the accuracy of classification of AD versus non-AD dementia cases (Parkinson's disease) by different machine leaning model when added with the VAE feature extraction and using most advanced machine learning models (Ridge Classifier, Light Boosting Machine, Extra Tree Classifier).

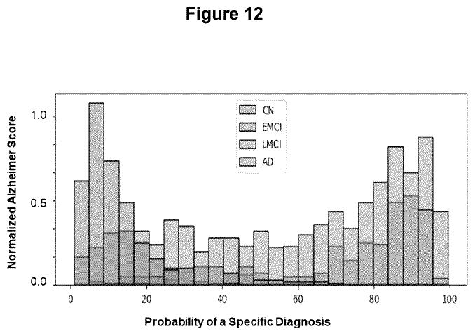

FIG. 12 illustrates the probability of calculating a specific diagnosis, Alzheimer's disease (AD), Late Mild Cognitive Impairment (LMCI), Early Cognitive Impairment (EMCI), and non-Demented Control (CN) in terms of Alzheimer Score. The machine learning model predicts a specific Alzheimer Score from an MRI scan.

### Tables

TABLE 1
Clinical diagnosis of non-demented control (CN), Alzheimer's
disease (AD), Mild Cognitive Impairment (MCI), Late MCI
(LMCI), Early MCI (EMCI) based on Mini-mental score examination
(MMSE), and Clinical Dementia Rating (CDR).

| Diagnosis | MMSE | CDR |
|---|---|---|
| CN | 29 and 30 | 0.0 |
| AD | <25 | >0.5 |
| LMCI | 25 and 26 | 0.5 |
| EMCI | 27 and 28 | 0.5 |

TABLE 2
Data source Alzheimer's Disease Neuroimaging Initiative (ADNI)

| | | Number of MRI |
|---|---|---|
| Diagnosis | Number of subjects | scans |
| CN | 180 | 344 |
| LMCI | 336 | 861 |
| AD | 148 (Autopsy- | 389 (Autopsy- |
| | Confirmed: 14) | Confirmed: 44) |

TABLE 3
Summarized classification by the current algorithm
using three Machine Learning models in combination
with Variational Autoencoder (VAE)

| | Patient | Accuracy | Precision | F1-Score | Recall |
|---|---|---|---|---|---|
| Algorithm | combination | (%) | (%) | (%) | (%) |
| VAE + | CN vs | 79.59 | 77.46 | 78.57 | 79.71 |
| Ridge | all-comers AD | | | | |
| Classifier | CN vs LMCI | 75.10 | 87.84 | 81.25 | 75.58 |
| | LMCI vs | 66.80 | 84.39 | 73.14 | 65.70 |
| | all-comers AD | | | | |
| | CN vs autopsy- | 91.03 | 95.38 | 94.81 | 92.75 |
| | confirmed AD | | | | |
| VAE + | CN vs | 84.35 | 82.86 | 83.45 | 84.06 |
| LGBM | all-comers AD | | | | |
| Classifier | CN vs LMCI | 73.44 | 79.67 | 81.92 | 84.30 |
| | LMCI vs | 78.80 | 84.39 | 84.64 | 84.88 |
| | all-comers AD | | | | |
| | CN vs autopsy- | 87.18 | 95.38 | 92.54 | 89.86 |
| | confirmed AD | | | | |
| VAE + | CN vs | 81.63 | 80.00 | 80.45 | 81.16 |
| ET | all-comers AD | | | | |
| Classifier | CN vs LMCI | 71.78 | 80.95 | 80.00 | 79.07 |
| | LMCI vs | 67.20 | 82.14 | 73.72 | 81.16 |
| | all-comers AD | | | | |
| | CN vs autopsy- | 79.49 | 94.92 | 87.50 | 81.16 |
| | confirmed AD | | | | |

AD: Alzheimer's Disease;
All-comers AD: Autopsy-confirmed AD + non-autopsy-confirmed AD;
CN: non-demented Control;
ET: Extra Tree;
LGBM: Light Gradient Boosting Model;
LMCI: Late Mild Cognitive Impairment

TABLE 4
Best algorithm for specific classification

| | | Accuracy of the |
|---|---|---|
| Algorithm | Patient combination | classification (%) |
| VAE + Ridge | CN vs LMCI | 75.10 |
| Classifier | CN vs autopsy-confirmed AD | 91.03 |
| VAE + LGBM | CN vs all-comers AD | 84.35 |
| Classifier | LMCI vs all-comers AD | 78.80 |

TABLE 5
Classification of current and future state of patients
by Mixture of Class Restricted Boltzmann Machines
Current Classifications

| Current | Precision (%) | Recall (%) | F1 score (%) | Accuracy (%) |
|---|---|---|---|---|
| CN | 100 | 86 | 92 | 86 |
| MCI | 100 | 82 | 90 | 82 |
| AD | 100 | 93 | 97 | 93 |

Future Predictions

| | | Precision | Recall | F1-score | Accuracy |
|---|---|---|---|---|---|
| Future | Current | (%) | (%) | (%) | (%) |
| CN | CN | 100 | 42 | 59 | 79 |
| | MCI | 70 | 100 | 82 | |
| | AD | 82 | 100 | 90 | |
| MCI | CN | — | — | — | 80 |
| | MCI | 84 | 80 | 82 | |
| | AD | 94 | 79 | 86 | |
| AD | CN | — | — | — | 100 |
| | MCI | — | — | — | |
| | AD | 100 | 100 | 100 | |

AD: Alzheimer's Disease;
CN: non-demented Control;
LMCI: Late Mild Cognitive Impairment

What is claimed:

1. A method of diagnosing Alzheimer's disease in human subjects comprising the steps of
(a) Variational Autoencoder (VAE) extracts relevant features from sMRI scans of subjects using a self-supervised learning methods (e.g., Ridge Classifier, Extra Tree; Light Gradient Boosting Model, and others),
(b) The model outputs a conditional transition probability matrix (cTPM) that gives both the current stage and probabilistic progression from present to future stages of dementia,
(c) Mixture of Class Restricted Boltzmann Machine in machine learning features extracted gives as an output a conditional transition probability matrix which provides the complete picture of future progression probabilities of mild cognitive impairment to be converted to Alzheimer's disease,
(d) The extracted feature vectors are then used as input conditions to different advanced machine learning classifications for diagnosing and predicting Alzheimer's disease.

2. The method of claim 1, wherein diagnosis is confirmed using at least one additional diagnostics step and non-Alzheimer's dementia.

3. The method of claim 1, wherein the underlying logic is that the algorithm is seen as a series of mathematical steps and together to achieve better performance in the invented algorithm.

4. The method of claim 2, wherein the new algorithm is superior to predicting and diagnosing Alzheimer's disease in comparison to other machine learning approaches.

5. The method of claim 1, wherein prediction of Alzheimer's progression leveraging neuroimaging data over multiple time steps.

6. The method of claim 1, wherein Variational Autoencoder-based dimensionality reduction generates a generative latent distribution, i.e., a probability distribution forms conditional probability distribution, can mitigate class imbalance.

7. The method of claim 1, wherein the mixture model architecture of class Restricted Boltzmann Machine captures distinct parameters characterizing the progression along three separate classes of dementia (Mild cognitive impairment, Alzheimer's disease, non-Alzheimer's, like dementia due to Parkinson's disease, frontotemporal dementia, vascular dementia, Lewy body dementia).

8. The method of claim 1, wherein the Mixture of Class Restricted Boltzmann Machine ML being a graphical model is robust to any encoded data, for instance, image, text, numeric, domain knowledge, can be converted to edges/connections between a set of variables in the graphical structure.

9. The method of claim 1, wherein the mixture of class Restricted Boltzmann Machine is a novel model currently applied for the prediction of Alzheimer's dementia; the model can be extrapolated to various other similar problem statements with minor to no tunings in the model.

10. The method of claim 1, wherein stacking multiple layers in the mixture model can also be used for the classification and exploration of intermediate stages; for example, for Alzheimer's disease, the mixture is restricted to 3 layers, each layer respectively corresponding to Control, Mild Cognitive Impairment, and Alzheimer's disease.

11. The method of claim 1, wherein using the same model with more layers, shows possible division in Mild Cognitive Impairment (MCI) to MCI-conversion and MCI-non-conversion.

12. The method of claim 1, wherein prediction of Alzheimer's progression leveraging neuroimaging data over multiple time steps.

13. The method of claim, wherein VAE-based dimensionality reduction generates a generative latent distribution, i.e., a probability distribution, and sampling from this conditional probability distribution can mitigate class imbalance.

14. The method of claim, wherein the mixture model architecture of class-RBMs captures.

15. The method of claim, prediction of Alzheimer's progression leveraging neuroimaging data over multiple time steps.

16. The method of claim, VAE-based dimensionality reduction generates a generative latent distribution i.e., a probability distribution. Sampling from this conditional probability distribution can mitigate class imbalance.

17. The method of claim, the mixture model architecture of class-RBMs captures distinct parameters characterizing the progression along three separate classes of dementia (CN, MCI, and AD).

18. The Method claim, the mixture of class-RBMs being a graphical model is robust to any encoded data for instance image, text, numeric, domain knowledge can be converted to edges/connections between a set of variables in the graphical structure with extrapolated to various other similar problem statements with minor to no tunings in the model.

19. The Method claim, the probability of a specific diagnosis, Alzheimer's disease (AD), Late Mild Cognitive Impairment (LMCI), Early Cognitive Impairment (EMCI), and non-Demented Control (CN) in terms of Alzheimer Score.
